(19) United States
(12) Patent Application Publication
(10) Pub. No.: US 2025/0011753 A1
(43) Pub. Date: Jan. 9, 2025

(54) METHOD AND TEST KIT FOR THE COST-EFFECTIVE AND RESOURCE-SAVING EXTRACTION OF NUCLEIC ACIDS

(71) Applicant: IST Innuscreen GmbH, Berlin (DE)

(72) Inventor: Timo Hillebrand, Hoppegarten (DE)

(73) Assignee: IST Innuscreen GmbH, Berlin (DE)

(21) Appl. No.: 18/710,265

(22) PCT Filed: Nov. 21, 2022
(86) PCT No.: PCT/EP2022/082644

§ 371 Date: Jul. 19, 2024

(30) Foreign Application Priority Data
Nov. 19, 2021 (DE) 10 2021 130 283.8

(51) Int. Cl. C12N 15/10; B01L 3/00

(52) U.S. Cl. CPC C12N 15/1006; B01L 3/5021; B01L 2300/0858; B01L 2400/088

(57) ABSTRACT

A method and test kit provide for the cost-effective and resource-saving extraction of nucleic acids. “Resource-saving” is to be understood as largely avoiding plastic waste. The method includes adding a binding buffer to a nucleic acid-containing solution where the nucleic acids are bound to a solid phase, washing the bound nucleic acids, and eluting the washed nucleic acids. A reaction vessel having grooves on or a thread cut into its inner side is used as the solid phase. Before the nucleic acids are bound, they can be released from cells by means of lysis. The test kit includes at least one reaction vessel as described, at least one binding buffer for binding the nucleic acids to the inner side of the reaction vessel, at least one wash buffer for washing the bound nucleic acids, at least one elution buffer for eluting the washed nucleic acids and optionally lysis buffers.

The invention relates to a novel agent for resource-saving, rapid and easily realizable isolation of nucleic acids from nucleic acids containing different starting materials which guarantees both a very high quality of the nucleic acids to be isolated and also enables the isolation of very high nucleic acid yields and, in addition, also enables the extraction of high molecular weight DNA due to the gentle execution.

### STATE OF THE ART

The most frequently performed extraction of nucleic acids from samples containing nucleic acids worldwide is well known to the person skilled in the art and is based on the binding of nucleic acids to mineral carriers in the presence of solutions of different chaotropic salts, in which finely ground glass powder (BIO 101, La Jolla, CA), diatomaceous earth (Sigma) or silica gels and/or silica suspensions or glass fiber filters or mineral earth (DE 41 39 664 A1; U.S. Pat. No. 5,234,809: WO-A 95/34569 DE 4321904; DE 20207793) are used as carrier materials. All these technical solutions are based on the binding of nucleic acids to a mineral carrier material based on glass or silicon in the presence of chaotropic salt solutions. Furthermore, the binding of nucleic acids to mineral solid phases can also be achieved very efficiently by means of so-called antichaotropic salts as a component of lysis/binding buffer systems (EP 1135479). And it is also possible to isolate nucleic acids with buffers containing a combination of chaotropic and non-chaotropic salts, as these buffers also provide the binding of nucleic acids to mineral carriers. In summary, the state of the art can therefore be described as follows: nucleic acids bind to mineral materials in the presence of buffers containing chaotropic or antichaotropic salts or also in the presence of buffers containing mixtures of chaotropic and antichaotropic salts and can then also be isolated in this way. There are also preferred variants in which aliphatic alcohols are also used providing binding. The person skilled in the art is also aware that all current commercial products for the isolation and purification of nucleic acids are based on this principle. The mineral carriers used are available in the form of loose fillings, filter membranes or suspension in this case. Paramagnetic or magnetic particles are often used to carry out automated extraction processes. These are, for example, silicate materials that have a magnetic or paramagnetic core or iron oxide particles the surface of which is modified in such a way that they carry the functionalities required for binding the nucleic acids. The extraction procedures for the isolation of nucleic acids are also based on the same principles: lysis of the starting sample to release the nucleic acid, binding of the nucleic acid to the corresponding mineral carrier material, washing of the bound nucleic acid, drying of the carrier material and final elution of the bound nucleic acid from the carrier material. Even though these procedures have proven their worth, there is, however, also a number of disadvantages. Thus, the binding capacity of the materials used is limited, especially when using spin filter membranes. If the initial sample contains too much nucleic acid, membranes often become clogged. The automated process sequences using magnetic particles are complex and, depending on the application, take a relatively long time to complete. It is not easy to carry out nucleic acid isolation under field conditions. It is also important to note that manual spin filter-based nucleic acid extraction in the laboratory in particular generates a large amount of plastic waste per preparation. Patent application WO 2016/169677 A1 discloses a completely different method for extracting nucleic acids. Mineral carrier materials are not used here. The nucleic acids are isolated using rough surfaces. Material with a rough or structured surface is used to bind the nucleic acids. In WO 2016/169677 A1, this material is always added to the reaction vessel in which the binding of the nucleic acids takes place, or is already there.

The prior art also includes the publications WO 2016/169679 A1 and WO 2016/169678 A1. The binding of nucleic acids to rough or “structured” material is also disclosed there. But the material responsible for the binding is surrounded by a liquid or is located on the outside of pipette tips.

A material that has grooves or a thread on the inside of a reaction vessel has not yet been described for binding nucleic acids.

### Object of the Invention

The invention was based on the task of improving the technical solutions of publications WO 2016/169677 A1, WO 2016/169679 A1 and WO 2016/169678 A1 with regard to the plastic waste problem.

### Solution of the Problem

The problem was solved in accordance with the features of the patent claims. In accordance with the invention, a method and a test kit were provided which enables the reaction process in a reaction vessel with grooves or a thread on the inside thereof.

The present invention is based on the task of providing an agent for extracting nucleic acids which allows high-quality nucleic acids to be isolated quickly and easily from samples containing nucleic acids, thereby drastically reducing the amount of plastic waste previously produced in the laboratory. The novel agent is based on the disclosed findings of patent application WO 2016/169677 A1 and solves the problem in an ideal way. Conventional plastic vessels (1.5 ml. 2.0 ml) are preferably used, although vessels for larger volumes (15 ml or 50 ml) could also be used. A thread is cut into the vessels using a thread cutter. Said thread is located in the lower half of the reaction vessel. The thread is preferably not all the way down to the bottom of the vessel, but slightly above the bottom of the vessel. A few thread turns are sufficient here. In the case of commercially available 1.5 ml reaction vessels, the thread is cut with a thread cutter, preferably of size M7, and in the case of commercially available 2.0 ml reaction vessels, a thread cutter, preferably of size M19, is used. The vessels modified by the inserted thread are now excellently suited for the extraction of nucleic acids. In a special embodiment, the entire extraction process from lysis to final desorption of the nucleic acids can be carried out in just one vessel. This significantly reduces the consumption of plastic and the plastic waste generated by conventional extraction methods. The extraction of nucleic acids follows the procedure that is well known to the person skilled in the art and thus follows the steps of lysing-binding-washing-eluting. In the case where free nucleic acids are present and the lysis step can be omitted, the steps are as follows: binding-washing-eluting-according to patent claim 1. With a lysis step, the extraction of nucleic acids from cells using the agent according to the invention is as follows: The cells are placed in the reaction vessel with the thread and lysed using a lysis buffer and with the addition of proteinase K from a commercially available kit (Smart DNA Mini Kit. IST Innuscreen GmbH). After cell lysis, isopropanol and Binding Optimizer (also from the kit) are added to the vessel with the lysate. The vessel is incubated on a shaker for 5 min at 1500 rpm. After this step, the sample is poured off. The nucleic acid is now on the thread. The vessel is then washed several times with alcoholic buffers. After ethanol removal, the bound nucleic acid is removed from the thread by adding water or a low salt buffer and is available for further applications. The process is particularly gentle for the extraction of DNA. This makes it possible to isolate high molecular weight DNA. There are no limitations with regard to binding capacities as is the case with spin filter or magnetic particle based extractions. No centrifugation and no magnetic particle separation is necessary, so no equipment for it is required either. In addition, only one vessel is used for the extraction described as an example. But alternatively, it is also possible to carry out the lysis step in an additional vessel and only switch to the vessel according to the invention after lysis has been completed. This is indicated if the samples are not completely lysed and therefore require a centrifugation step in order to separate insoluble components from the lysate.

The reaction vessels according to the invention with grooves or a thread on the inside are preferably made of plastic. But other materials, which are equipped with grooves or a thread, can also be used.

Conserving resources in the sense of this invention means avoiding plastic waste.

The invention is described below by means of examples, wherein the examples do not imply any limitation of the applications.

### EMBODIMENTS

### Example 1: DNA Extraction from Nucleated Blood Cells Using a 1.5 ml Reaction Vessel with a Thread Cut into the Reaction Vessel in Comparison with Two Commercially Available Reference Extraction Kits Based on Spin Filter Columns (Qiagen; DNeasy Blood & Tissue Kit and IST Innuscreen GmbH Innuprep DNA Mini Kit 2.0)

Whole blood samples of 3 ml were used to isolate the nucleated cells from them. These cells were then used for the extraction. The extractions with the two commercially available products were carried out according to the user manual. The extraction using the agent according to the invention was carried out as follows. All necessary reagents from a likewise commercially available kit (IST Innuscreen GmbH: SmartBlood DNA midi Kit (m)) were used in the process. The cells were resuspended in 120 μl 1×PBS buffer and transferred to the reaction vessel according to the invention. After the addition of 200 μl lysis buffer and 30 μl proteinase K, the reaction vessel was incubated in a thermoshaker at 55° C. for 30 min. After cell lysis. 350 μl isopropanol and 40 μl Binding Optimizer were added to the reaction vessel. The vessel was then incubated for 5 min on a shaker at 1500 rpm. The sample was then poured off. The DNA bound to the thread was washed three times with an alcoholic wash buffer. After the last washing step, the vessel was incubated at 40° C. for 10 min with the lid open. Finally, 300 μl of an elution buffer (10 mM Tris-HCl) was added to the vessel and the DNA was dissolved at 50° C. for approx. 30 min. Subsequently, the DNA was measured spectrophotometrically and visualized on an agarose gel. This was also done with the extracted DNA samples from the two reference kits.

TABLE 1
Table 1 shows the spectrophotometric measurement of the DNA.

| | | Yield | | |
|---|---|---|---|---|
| Sample | Type of Sample | (μg) | A₂₆₀:A₂₈₀ | A₂₆₀:A₂₃₀ |
| 1 | Reference Kit Qiagen; sample 1 | 32 | 1.9 | 2.1 |
| 2 | Reference Kit Qiagen; sample 2 | 31 | 1.9 | 2.1 |
| 3 | Reference Kit IST Innuscreen; | 34 | 1.9 | 2.0 |
| | sample 1 | | | |
| 4 | Reference Kit IST Innuscreen; | 33 | 19 | 2.2 |
| | sample 2 | | | |
| 5 | Agent according to the | 82 | 1.9 | 2.1 |
| | invention; sample 1 | | | |
| 6 | Agent according to the | 79 | 1.9 | 2.1 |
| | invention; sample 2 | | | |

### Example 2: DNA Extraction from Nucleated Blood Cells Using a 1.5 ml Reaction Vessel with a Thread Cut into the Reaction Vessel in Comparison with the Commercially Available Reference Extraction Kit (Qiagen; DNeasy Blood&Tissue Kit) in Terms of the Amount of Plastic Waste Generated

This was based on 100 reactions. For this, the reference kit requires 100 reaction vessels for sample lysis, 100 spin filter columns, 300 collection vessels for the spin filter column and 100 reaction vessels for collecting the eluted DNA. The method using the agent according to the invention requires 100 threaded reaction vessels.
Amount of plastic waste reference kit (100 reactions): 0.6 kg
Amount of plastic waste agent according to the invention (100 reactions): 0.1 kg

This means that extraction using the agent according to the invention is significantly more resource-saving and leads to a drastic reduction in laboratory waste.

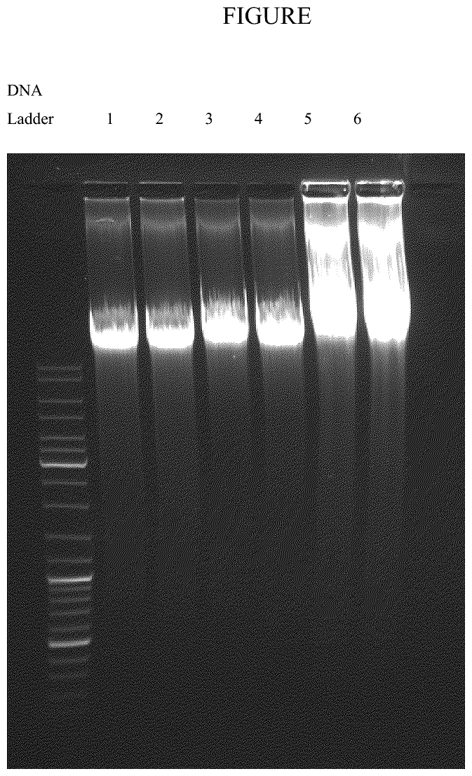

FIG. 1 shows the gel electrophoretic analysis of the DNA (0.8% agarose gel) for example 1. The data show that the DNA yield extracted with the agent according to the invention is significantly higher than the yield obtained with the two reference kits based on filter columns.

1. A method for resource-saving extraction of a nucleic acid, comprising:
a) adding a binding buffer to a solution containing said nucleic acid, wherein said nucleic acid is bound to a solid phase,
b) washing of the bound nucleic acid, to obtain a washed nucleic acid and
c) eluting of the washed nucleic acid
wherein a reaction vessel serves as the solid phase which has grooves or a
thread cut into an inner side to which the nucleic acid is bound.

2-6. (canceled)

7. The method according to claim 1, wherein prior to a), a lysis step and, optionally, a centrifugation takes place.

8. The method according to claim 1, wherein the reaction vessel comprises a plastic.

9. The method according to claim 1, wherein the grooves or the thread are in a lower part of the reaction vessel.

10. A test kit for carrying out the method according to claim 1, the test kit comprising:
a) at least one reaction vessel having grooves or a thread inside,
b) at least one binding buffer for binding the nucleic acid to the inside of the reaction vessel,
c) at least one wash buffer for washing the bound nucleic acid, and
d) at least one elution buffer for eluting the washed nucleic acid.

11. The test kit according to claim 10, further comprising:
at least one lysis buffer.
